(19) United States
(12) Patent Application Publication
(10) Pub. No.: US 2023/0290521 A1
(43) Pub. Date: Sep. 14, 2023

(54) On-demand service application that will allow allied healthcare professionals to work independently and connect with individuals, businesses, doctors, and laboratories that are in need of at-home allied healthcare services

(71) Applicant: CHIMENE BARNETT, WOODSTOCK, GA (US)

(72) Inventor: CHIMENE BARNETT, WOODSTOCK, GA (US)

(21) Appl. No.: 17/691,810

(22) Filed: Mar. 10, 2022

(51) Int. Cl. G16H 80/00; G16H 40/20; G16H 40/67; G16H 10/40; H04W 4/029

(52) U.S. Cl. CPC G16H 80/00; G16H 40/20; G16H 40/67; G16H 10/40; H04W 4/029

(57) ABSTRACT

The use of on-demand services has increased over the past couple of years. Examples of on-demand services include, but are not limited to, taxi services, food delivery, grocery services, etc. Direct Paramed Mobile Health Solutions mobile application will allow businesses, healthcare organizations, and individuals to directly connect with credentialed allied healthcare professionals on-demand. It eliminates the traditional appointment-based methodology, enabling healthcare professionals to work independently and connect with businesses, doctors, and laboratories in need of essential healthcare workers while building entrepreneurship. In contrast, it allows an individual peace of mind to be seen at home or in their office without the need for scheduling an appointment or going to a facility.

### BACKGROUND

As Healthcare Professionals struggle to keep up with the demands of today's healthcare system, individuals, laboratories, physicians, and companies have begun working directly with allied healthcare professionals to enhance the value and efficiency of both lab tests and laboratory-based services. Direct Paramed Mobile Health Solutions gives individuals, laboratories, physicians, and businesses the ability to order on-demand healthcare services from the comfort of their homes or office. Eliminating the hassle and inconvenience of scheduling separate lab visits to a lab or hospital facility, and can also eliminate the need to travel to an outside lab.

In the era of consumer-driven same-day access to allied healthcare laboratory services, the customer will have the ability to order laboratory tests through Direct Paramed Mobile Health Solutions application with a credentialed professional arriving in minutes if available within the vicinity. For healthcare facilities, this will eliminate the revolving door of consistently hiring new staff and allow a convenient way for businesses to request allied healthcare services such as pre-employment drug screening services on-demand. This mobile application will enhance the User's business aspect and allow people with limitations or immune-compromised fast access to extended laboratory services without leaving their home or office.

### SUMMARY

The invention can be implemented in numerous ways, including as a process; a system; a computer program product embodied on a computer readable storage medium; and/or a processor, such as a processor configured to execute instructions stored on and/or provided by a memory coupled to the processor. In this specification, the order of the steps of disclosed processes may be altered within the scope of the invention.

The service system includes a service provider server system application executed by mobile device in wireless communication over a network with at least one service requesting application, wherein the requesting application is executed by a mobile device or computer system. The requesting application includes a request for services such as phlebotomy services, urine collection, biometric screening, and any additional allied healthcare services.

Instant notification is sent to the surrounding providers within a predetermined radius by the system. Surrounding providers have the capability to accept or decline requested services. Both customer and accepting provider can communicate securely using our designated application programming interface via phone or text message once service is accepted.

In the event there is no provider in the area, the system will send a notification to the requesting customer within 30 minutes of placing the original request.

Although multiple providers in the area may receive a notification of service requests, only one provider is able to accept the service upon a provider accepting the request. The requesting application is configured, on a request by a user of the application, to request a service by transmitting the geographical position (GPS) of the services address with an indication of the requested service to the service provider application server-system. Providers have 24 hours to transport the obtained specimen to the respective laboratory.

To ensure proper specimen handling, the provider service system application has multiple action buttons that the provider cannot pass until completed, sending the customer status notifications of each phase until the designated laboratory receives the specimen. The provider does not receive payment until confirmed receipt by the respective laboratory.

### DETAILED DESCRIPTION OF THE INVENTION

A detailed description of one or more embodiments of the invention is provided below, along with accompanying figures that illustrate the principles of the invention. The invention is described in connection with such embodiments, but the invention is not limited to any embodiment. The scope of the invention is limited only by the claims, and the invention encompasses numerous alternatives, modifications, and equivalents. Numerous specific details are outlined in the following description to provide a thorough understanding of the invention. These details are provided, for example, and the invention may be practiced according to the claims without some or all of these specific details. For the purpose of clarity, technical material that is known in the technical fields related to the invention has not been described in detail so that the invention is not unnecessarily obscured.

The Version of The Invention Discussed Here Includes:

REQUESTING CUSTOMER (Android & iOS web-based capability)
1. Customer will download the application and create an account and login User sign up/login by the following process:
   Splash Screen
   Auto Detection location
   Enter Phone Number
   OTP Verification
   Manual Sign Up
   Manually set location
   Social media login (Google, Facebook)
   Forgot Password/Reset Password
   Login as a guest.
2. Customer will enter the donor information: Name, Address, Phone number and any special notes for the care provider Search Service Screen
   Service listing:—Users can view services listings with name and image
   Add donor service listing details: Final User will add details related to the donor.
   Upload documents:—final User can upload documents if required to request a specific service.
3. Customer will choose a service option: Blood work, EKG, Vital, Urine, DOT/non-DOT Drug Screen, Blood Alcohol Testing, Paramedical Exam etc. Add service Screen and Place Request Screen:—
   Service Name:—Users can view the service name
   Continue button:—Click on the continue button user can move on the place service screen.
   Enter a service donor address or change address
4. Customer will choose option to pay
   Apply coupon: —Users can also apply promo code.
   Bill Detail: —Users can view the total services, sales tax, service, service fee, tip, and total payment amount.
   Proceed to pay: —Users can select the online payment option or bill.
   Request services: —After placing requested services, the User will get the button of “Go To Request”. Click on this button, the User can view the service in detail.
   Once the service is requested is accepted by the service provider, the User will get the notification for service confirmation
Service Confirmation: —User will get the message with a service detail, service provider name, contact Information, service timing, and location as well, the User can track the requested services.
   Cancel Services
   Live Tracking
   Service completed confirmation
   Rating and Review: —Users can give the review to the service provider
5. Customer Requested Services Screen: Requested Services Screen (History Of service):—In the requested service screen the User can view the upcoming and past services.
   Upcoming Services: —On upcoming service request, the User can view the services requested that he has placed recently but not completed, the User can view services detail with service name, price etc. . . . .
   Past services: —On a past service request users can view the listing of past services, with services, name, price, date, and status of the service Cancelled/Accepted) click on a particular service user can see the complete service information.
6. Account Screen
   Profile: —On a profile view user can view the image, name, email Id, phone no and location as well he can edit the profile and change the password.
   Invite Friend: —Users can also share the URL for inviting friends.
   My Address: —View and edit address
   Payment Detail: —Add and delete the payment method and billing details
   Help & Support: —Users can see some by default questions/answers regarding the service request, and payment.
      If the User has any other query, he can click on the button “Send A Message”, click on this button, the User can fill the form “Enter Name, Message and Submit Button”, This query will submit to the support system, they will reply to user query answer.
7. Customer will submit Request
8. The information will send a message to the healthcare providers within the network in a predetermined radius
9. Someone will accept the request, GEO location system will activate allowing both customer, donor, and provider identify who's coming and estimate time of arrival
10. The customer or donor can write a review
   SERVICE PROVIDER PANEL (Android & iOS)
1. Sign Up/Login: Provider will need to upload their documents so admin can verify them and they can sign up/log in with the following process:
   Manual Sign Up:—First the provider needs to fill the required field for sign up, “user name, email Id, password, mobile number, and address”, click on the button, next form will be open for uploading the document.
   Upload certification/licensure: Once the following Information will be submitted and the profile will require approval upon completion of the vetting process.
2. Home Screen: —On the home screen, the service provider can view the map, and availability option button.
   Go Offline/Online: service providers can go Online & Offline by clicking on the availability button.
   Manage services Requests: The provider can view the latest service request with the approving and reject icon.
Note: —If a provider rejects a service, it must be sent to the next provider rather than canceling a service request.
   Accepting service request: —After accepting the request, the service provider will see the donor details and service address
   service donor location (Navigation):
   service donor location: —service provider can view the service's details, donor name, price, and service address with a phone Icon (Click on call icon, the service provider will be able to perform a call with the donor without disclosing the phone number.)
   View donor Map Location
   Navigate to Donor Location
   Specimen obtained or Unobtained
   Mark services as Complete
   View service History
3. Dashboard: —On a dashboard service provider can view the following:
   Documents: —service provider can add and delete current certification.
   History: —service provider can view the previous requested service history listing with date, time, service address, not the cost
   Earning
   Profile:
   Help and Support: —Provider can contact the super admin with an email and phone number, If he requires any help.

### Embodiment Details

User A: Customer
User B: Service provider(s)

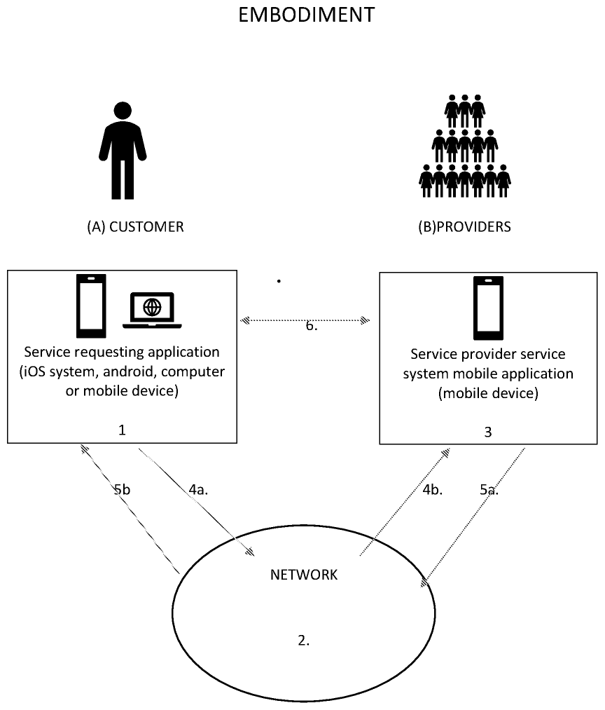

FIG. 1 Service requesting application (iOS system, android, computer or mobile device)

FIG. 2. Network

FIG. 3. Service provider service system mobile application (iOS, android or mobile device)

FIG. 4a. Service request sent from service requesting application

FIG. 4b. Service request notification received to all available providers within radius of service address

FIG. 5a. Service provider service application transmits notification(s) of status on requested order

FIG. 5b. Service requesting application receives update status on current request until completed

FIG. 6. Application Programming Interface which allows customer and service provider to communicate securely.

Invention claimed is:

1. A healthcare-based on-demand mobile service application system, comprising: service provider and customer in wireless communication over a network with multiple providers available to receive instant notification of healthcare service request, wherein the requesting application is executing in a mobile device or computer-based system, wherein the requesting application is configured, on a request by a user of the application, to request a healthcare service such as but not limited to phlebotomy services, urine collection, biometric screening, blood pressure, electrocardiograph, life insurance medical exam, and any additional allied healthcare services. By transmitting a map information comprising a visual symbol located on the geographical position (GPS) of the service address entered by the customer of service location wherein a customer can request services for someone at a different location other than the location of the ordering device: together with an indication of the requested service to the service provider server-system, the service provider server-system contacts all providers online in radius of the service address entered by the customer, only one service provider of the indicated requested service located within the requested radius can accept the service, service providers respond to the request of service by sending “accept” notification and map information layer to the customer server-system, wherein the submitted map information layer comprises a visual symbol on the geographical position of the service provider, the service provider server-system submits respective received map information layers to the requesting customer device, wherein the customer can track the location and arrival time of location of the accepting service provider. Thereby a communication link between the User is established over the network; wherein the service provider server system submits notification to ordering customer with updates and specimen tracking detail after the services have been rendered; wherein the customer server system will automatically send a provider rating notification to maintain high quality of service which allows the customer to rate the provider on attire, performance, and professionalism.

2. The system of claim 1, wherein multiple healthcare service providers within a radius of the geographical location of the requesting customer's service location entered in the customer server application, will receive instant notification of healthcare services.

3. The system of claim 2, wherein a customer or service provider can upload documents and pictures to notify the respective parties of services requested and status updates of respective rendered services.

4. The system of claim 3, wherein a user and service provider will receive a map of geographical positions of the service location and approaching provider location.

5. The system of claim 1, wherein the service is an on-demand healthcare-related service application consisting of but not limited to phlebotomy, urine collection, electrocardiography, and biometric screening.

6. The system of claim 1, wherein the service location is determined by the service address entered by the ordering customer.

7. The system of claim 2, wherein the requesting application will receive notifications updating when the specimen is obtained or not obtained.

8. The system of claim 1, wherein the service provider server-system is configured to request a user authentication of a service provider when accepting services.

9. The system of claim 1, wherein a default radius within the service provider server-system application is defined by the geographical distance from the service address entered of the service requesting application.
